(19) United States
(12) Patent Application Publication
(10) Pub. No.: US 2018/0261309 A1
(43) Pub. Date: Sep. 13, 2018

(54) METHODS AND SYSTEMS FOR ESTIMATING COSTS OF PERINATOLOGICAL OR NEONATOLOGICAL CARE

(71) Applicant: MEDNAX Services, Inc., Sunrise, FL (US)

(72) Inventor: Ingrid Vasiliu-Feltes, Sunrise, FL (US)

(21) Appl. No.: 15/918,506

(22) Filed: Mar. 12, 2018

Related U.S. Application Data
(60) Provisional application No. 62/470,596, filed on Mar. 13, 2017.

(51) Int. Cl. G16H 15/00; G16H 50/70; G16H 50/30

(52) U.S. Cl. CPC G16H 15/00; G16H 50/30; G16H 50/70

(57) ABSTRACT

Methods and systems for estimating costs of perinatological or neonatological care are provided. An example system comprises a communication module for receiving historical perinatal data and beneficiary perinatal data. The system also includes a classification module to determine whether the historical perinatal data has met predetermined criteria. Based on the determination, the classification module selectively analyzes the historical perinatal data and the beneficiary perinatal data and attributes a beneficiary to at least one perinatal care episode based on the analysis. A scoring module of the system ascertains variables for calculation of a risk score associated with the perinatal care episode, determines coefficients corresponding to the variables, calculates the risk score for the beneficiary based on the coefficients, and assigns the beneficiary a financial risk tier based on the risk score for the perinatal care episode. The system may also include a payment calculation module and a virtual interface module.

### CROSS-REFERENCE TO RELATED APPLICATIONS

This application claims the benefit of U.S. provisional patent application Ser. No. 62/470,596 filed Mar. 13, 2017, entitled “Systems and Methods for A Neonatology and Perinatology Total Cost of Care Alternative Payment Model Technology Platform,” which is incorporated herein by reference in its entirety for all purposes.

### TECHNICAL FIELD

This disclosure generally relates to health care data processing. More specifically, this disclosure relates to estimating costs of perinatological and neonatological care.

### BACKGROUND

Existing solutions for healthcare cost estimation and analysis use isolated data sets (e.g., payer data, claims data, or clinical data) or data set combinations that are analyzed in an asynchronous fashion. Moreover, existing solutions concerned with healthcare data for adult or pediatric beneficiaries do not work well with neonatological or perinatological beneficiaries due to small data sets and insufficient consideration for maternal and pre-natal risk factors.

### SUMMARY

This section introduces a selection of concepts in a simplified form that are further described in the Detailed Description section, below. This summary does not identify key or essential features of the claimed subject matter and is not intended to be an aid in determining the scope of the claimed subject matter.

This disclosure is generally concerned with methods and systems for healthcare data processing, analysis, and visualization involving embodiments of estimating costs of perinatological or neonatological care. Although the following description primarily focuses on perinatology, neonatology, and related medicine, embodiments of this technology can also be applied to other healthcare fields.

The methods and systems presented in this disclosure are directed to estimating healthcare costs and optimizing quality-to-cost ratios for most vulnerable patients, premature infants, and mothers having a high risk of developing complications or delivering premature infants. The present technology not only helps healthcare providers, patients, and beneficiaries to find optimal healthcare solutions, but also improves healthcare quality and treatment outcomes and provides optimal and efficient healthcare and coordination between various participants in the healthcare field. Below are some embodiments of the present disclosure, while other embodiments should be evident from the following detailed description of example embodiments, claims, and accompanying drawings.

According to one embodiment of this disclosure, there is provided a system for estimating a cost of perinatological or neonatological care. An example system comprises a communication module, a classification module, and a scoring module. The communication module is configured to receive historical perinatal data associated with a plurality of patients and beneficiary perinatal data associated with a beneficiary. The historical perinatal data and the beneficiary perinatal data can be derived from payer data, claims data, or clinical data sources. Notably, the communication module is agnostic to a data source associated with the historical perinatal data and the beneficiary perinatal data especially when formatted or normalized according to an industry accepted format.

The classification module is configured to determine that the historical perinatal data has met predetermined criteria which are associated with volume criteria, clinical hospital criteria, and clinical provider criteria. Based on the determination, the classification module selectively analyzes the historical perinatal data and the beneficiary perinatal data, and further attributes the beneficiary to at least one perinatal care episode based on the analysis.

The scoring module is configured to ascertain a plurality of variables for calculation of a risk score associated with the at least one perinatal care episode. Some examples of variables include a maternal age, a gestational age, a birthweight, ethnicity, a maternal risk factor, an infant risk factor, a maternal procedure, an infant procedure, a maternal lab, an infant lab, imaging, a medication, genetic testing, a diagnosis category, a Current Procedural Terminology (CPT) code, a cost of an outpatient treatment, a cost of an inpatient treatment, a cost of an emergency room treatment, and optionally other variables. The scoring module is further configured to determine a plurality of coefficients corresponding to the plurality of variables for at least one perinatal episode based on the beneficiary perinatal data, calculate the risk score for the beneficiary based on the coefficients, and assign the beneficiary a financial risk tier based on the risk score for the perinatal care episode. In some embodiments, the risk score can be calculated by multiplying two or more of the plurality of coefficients.

The system may also include an optional payment calculation module depending on an embodiment. The payment calculation module is configured to generate cost of care data for at least one perinatal care episode for the beneficiary based on the financial risk tier of the beneficiary and optionally other data such as a predetermined quality to cost ratio, a cost of a hospital service, or a cost of a physician service. The payment calculation module can also estimate a payment amount for a customer, such as a health plan, a hospital system, an actuary, or a provider group, based on the estimated cost of care.

The system may also include a virtual interface module configured to receive the cost of care data, customize the cost of care data for the customer based on predetermined customer criteria to generate customized cost of care data, and generate a customized report for the customer based on the customized cost of care data.

The modules of the system may be implemented using a variety of technologies. For example, the modules described herein may be implemented in software executing on a computer system or in hardware utilizing either a combination of microprocessors or other specially designed application-specific integrated circuits (ASICs), programmable logic devices, or various combinations thereof. As such, the modules of the system can be implemented within one computer or server or within multiple computers or servers connected into a network. Other hardware implementations may involve, partly or entirely, transistor-based circuits. For example, the communication module may involve a radio modem, Ethernet module, network interface, communication port, or circuit terminals. Other modules can have hardware implementation involving programmable and non-programmable microcontrollers, processors, circuits, computing devices, servers, and the like.

According to another embodiment of this disclosure, a method for estimating a cost of perinatological or neonatological care can be implemented by computer hardware, software, or any variations thereof. In another example, the method of this embodiment may be implemented by a series of computer-executable instructions residing on a transitory or non-transitory storage medium such as a disk drive or computer-readable medium.

An example method of this embodiment comprises: receiving, by a communication module, historical perinatal data associated with a plurality of patients; receiving, by the communication module, beneficiary perinatal data associated with a beneficiary; determining, by a classification module, that the historical perinatal data has met predetermined criteria; based on the determination, selectively analyzing, by the classification module, the historical perinatal data and the beneficiary perinatal data; based on the analysis, attributing, by the classification module, the beneficiary to at least one perinatal care episode; ascertaining, by a scoring module, a plurality of variables for calculation of a risk score associated with the at least one perinatal care episode; determining, by the scoring module, a plurality of coefficients corresponding to the plurality of variables for the at least one perinatal episode based on the beneficiary perinatal data; calculating, by the scoring module, the risk score for the beneficiary based on the coefficients; and assigning, by the scoring module, the beneficiary a financial risk tier based on the risk score for the perinatal care episode.

Additional objects, advantages, and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities, and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 shows a block diagram of an example computer system architecture suitable for implementing methods for healthcare cost estimation, according to one example embodiment;

FIG. 2 shows a high-level abstract diagram of a process for determining coefficients, according to one example embodiment;

FIG. 3 shows a high-level diagram of calculating a risk factor according to one example embodiment;

FIG. 4 shows a high-level representation of a risk score and respective financial risk tiers, according to one example embodiment;

FIG. 5 shows a high-level diagram of three risk tiers aligned with corresponding payment methodologies, according to one example embodiment;

FIG. 6 shows a high-level diagram of system for implementing methods for healthcare cost estimation involving data sources used to provide customized reports via a virtual interface module, according to one example embodiment;

FIG. 7 is a process flow diagram showing a method for estimating a cost of perinatological or neonatological care, according to an example embodiment; and

FIG. 8 is a computer system that may be used to implement the methods for estimating a cost of perinatological or neonatological care as described herein.

### DETAILED DESCRIPTION OF EXAMPLE EMBODIMENTS

The following detailed description of embodiments includes references to the accompanying drawings, which form a part of the detailed description. Approaches described in this section are not prior art to the claims and are not admitted to be prior art by inclusion in this section. The drawings show illustrations in accordance with example embodiments. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Embodiments of this disclosure are concerned with methods and systems for processing, analysis, and visualization of data associated with healthcare, and more particularly with perinatological or neonatological care. The methods described herein can be implemented by hardware modules, software modules, or a combination of both. The methods can also be embodied in computer-readable instructions stored on computer-readable media. As should be evident from the following description, the methods and systems of this disclosure allow for improving healthcare outcomes and reducing costs for neonates and mothers having high risk complication. The methods also provide unique total cost-of-care insights for healthcare costs, rather than value-based healthcare cost information, and address all components of the neonatology and perinatology fields: payer costs, hospital costs, physician costs, and patient costs. Furthermore, the methods use combined input data sets, rather than isolated input data sets, to estimate healthcare costs, which allows performing cost analysis for various recipients (such as a beneficiary, patient, healthcare provider, insurer, etc.) simultaneously. The methods also account for customized beneficiary attribution and correlation of risk scores with payment amounts. This approach allows for cost savings and an optimal quality-to-cost ratio.

The embodiments will now be presented with reference to the accompanying drawings. These embodiments are described and illustrated by various modules, blocks, components, circuits, steps, operations, processes, algorithms, and the like, collectively referred to as “elements” for simplicity. These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented with a “processing system” that includes one or more processors. Examples of processors include microprocessors, microcontrollers, Central Processing Units (CPUs), digital signal processors (DSPs), field programmable gate arrays (FPGAs), programmable logic devices (PLDs), state machines, gated logic, discrete hardware circuits, and other suitable hardware configured to perform various functions described throughout this disclosure. One or more processors in the processing system may execute software, firmware, or middleware (collectively referred to as “software”). The term “software” shall be construed broadly to mean instructions, instruction sets, code, code segments, program code, programs, subprograms, software components, applications, software applications, software packages, routines, subroutines, objects, executables, threads of execution, procedures, functions, and the like, whether referred to as software, firmware, middleware, microcode, hardware description language, or otherwise. The software may be stored on or encoded as one or more instructions or code on a non-transitory computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), compact disk ROM (CD-ROM) or other optical disk storage, magnetic disk storage, solid state memory, or any other data storage devices, combinations of the aforementioned types of computer-readable media, or any other medium that can be used to store computer executable code in the form of instructions or data structures that can be accessed by a computer.

For purposes of this document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

The term “client device” can refer to a personal computer, a laptop computer, tablet computer, smartphone, mobile phone, Internet phone, netbook, set top box, multimedia player, personal digital assistant, server computer, network storage computer, entertainment system, infotainment system, a television device, a display, or any other computing device comprising at least a networking and data processing capabilities.

The term “module” shall be construed to mean a hardware device, software, or a combination of both. For example, a hardware-based module can use either one or more microprocessors, ASICs, programmable logic devices, transistor-based circuits, or various combinations thereof. Software-based modules can constitute computer programs, computer program procedures, computer program functions, and the like. In addition, a module of a system can be implemented by a computer or server, or by multiple computers or servers connected into a network. Hardware or software implementations can depend on particular system implementation and constraints. For example, a communication module may include a radio modem, Ethernet module, network interface, communication port, or circuit terminals. In other embodiments, a communication module may include software, software procedure, or software-based function configured to receive and transmit data by a hardware device, such as a processor. Other implementations of communication module can involve programmable and non-programmable microcontrollers, processors, circuits, computing devices, servers, and the like.

Referring now to the drawings, exemplary embodiments are described. The drawings are schematic illustrations of idealized example embodiments. Thus, the example embodiments discussed herein should not be construed as limited to the particular illustrations presented herein, rather these example embodiments can include deviations and differ from the illustrations presented herein.

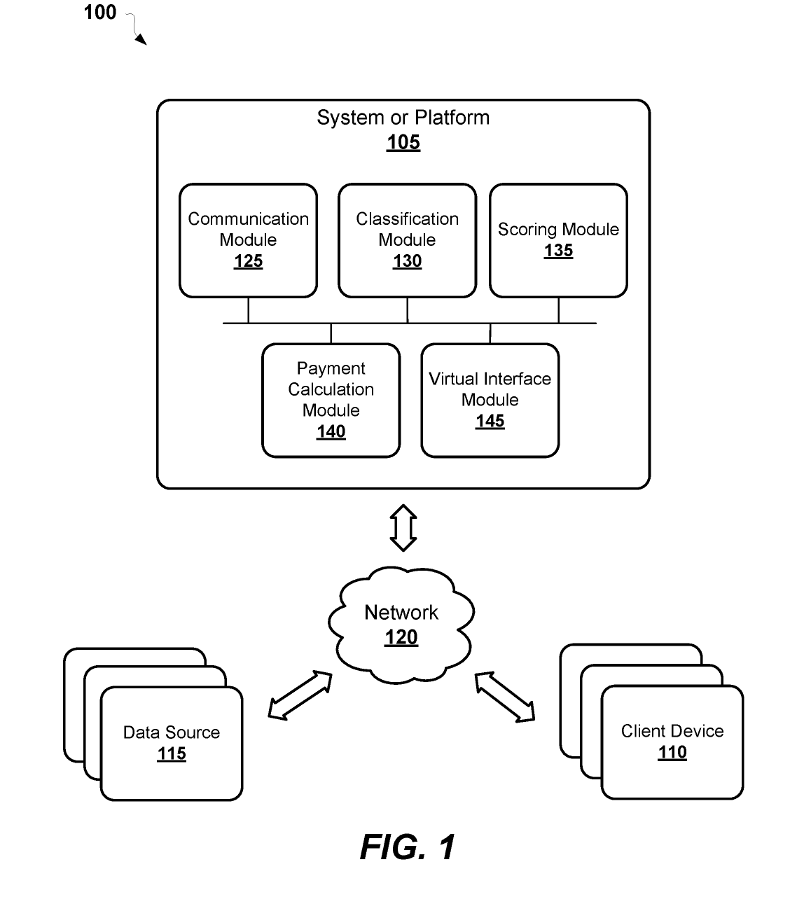

FIG. 1 shows a block diagram of an example system architecture 100 suitable for implementing methods for healthcare cost estimation according to one example embodiment. System architecture 100 includes a system (technology data processing platform) 105 configured to provide cost estimation of healthcare, particularly perinatological or neonatological care. As shown in the figure, system 105 includes various modules, which are discussed below, and also directly or indirectly connected to one or more client devices 110 via one or more communication networks 120. System 105 is also directly or indirectly connected to one or more data sources 115 via one or more communication networks 120. Client devices 110 can include personal computers, workstations, mobile devices, and the like, which are operated by users. The users can include customers, healthcare providers, patients, beneficiaries, insurers, health plan administrators, hospital system operators, actuaries, provider group administrators, and so forth. Data sources 115 can include one or more databases, data storages, memories, computer servers, file servers, networked devices, and the like. Data sources 115 can collect and store historical perinatal data and beneficiary perinatal data. Data sources 115 can also collect and store payer data, claims data, and clinical data. Data sources 115 can be independent and not associated with one another.

Communications network 120 refers to any wired, wireless, or optical networks including, for example, the Internet, intranet, local area network (LAN), Personal Area Network (PAN), Wide Area Network (WAN), Virtual Private Network (VPN), cellular phone networks (e.g., Global System for Mobile (GSM) communications network, packet switching communications network, circuit switching communications network), Bluetooth radio, Ethernet network, an IEEE 802.11-based radio frequency network, a Frame Relay network, Internet Protocol (IP) communications network, or any other data communication network utilizing physical layers, link layer capability, or network layer to carry data packets, or any combinations of the above-listed data networks.

System 105 includes a communication module 125 which is configured to receive and transmit data over communications network 120. More specifically, communication module 125 dynamically receives historical perinatal data associated with a plurality of patients from one or more data sources 115. The historical perinatal data can include claims data (e.g., diagnostic and procedure codes, All-Patient Refined (APR) Diagnosis Related Group (DRG) coefficients, Hierarchical Condition Category (HCC) coefficients, Centers for Medicare and Medicaid Services (CMS) coefficients, healthcare costs and charges, payments, billing codes, specialty care data, length of stay (LOS) data, mortality data, level of service data, etc.), clinical data, hospital services data (e.g., data associated with ancillary hospital services such as transfusions, central lines, ventilation, intravenous(IV) medications, imaging services, radiology, etc.), clinical data-provider services data (e.g., diagnosis and procedure codes, CPT codes , etc.), and/or research data (e.g., scales, scores, calculators, etc.). The historical perinatal data can also include information on treatment plans and procedures, treatment outcomes, costs, patient demographics and bibliographic information, patient histories, insurance data, healthcare provider data, and so forth. Communication module 125 also dynamically receives beneficiary perinatal data associated with a beneficiary from one or more data sources 115. The beneficiary can be a patient or patient's parent or guardian, or the beneficiary can be any other individual or organization. The beneficiary perinatal data can be of the same or similar type or format as the historical perinatal data.

In certain embodiments, the historical perinatal data and the beneficiary perinatal data are derived from payer data, claims data, or clinical data, which are collected in one or more data sources 115. Notably, communication module 125 is agnostic to data source 115 associated with the historical perinatal data and the beneficiary perinatal data, especially when the historical perinatal data and the beneficiary perinatal data are formatted or normalized according to an industry accepted format. For example, the historical perinatal data and the beneficiary perinatal data can be formatted by data sources 115 or system 105 to include data of medical procedures containing CPT codes, DRG codes, or International Classification of Diseases (ICD) codes.

System 105 further includes a classification module 130 which is configured to process and analyze the historical perinatal data and the beneficiary perinatal data. Particularly, classification module 130 determines that the historical perinatal data has met predetermined criteria. This determination can be based on at least one of the following: volume criteria (e.g., enough services for a condition associated with a specific type of CPT, DRG, or ICD code were provided during a performance period), clinical hospital criteria (e.g., specific procedures or ancillary services), and clinical provider criteria (e.g., specific services). The criteria can be predetermined by an operator of system 105, healthcare plan provider, healthcare provider, customer, and so forth. Based on the determination, classification module 130 selectively analyzes the historical perinatal data and the beneficiary perinatal data to attribute the beneficiary to at least one perinatal care episode. The perinatal care can include pre-natal care, neo-natal care, post-partum care, or pediatric care. The term “perinatal care episode” may refer to a category of healthcare and a medical condition. The episode may optionally involve one or more of the following: a treatment plan, medical and financial risks, possible complications, related stats, and so forth. For example, a perinatal care episode can be birth to post-natal day 45. The analysis performed by classification module 130 can involve statistical analysis, comparative analysis, detailed mapping, database correlation, modeling, validation, calibration, and other procedures. In some embodiments, a machine-learning algorithm can be used for that purpose.

System 105 further includes a scoring module 135 as shown in FIG. 1. Scoring module 135 is configured to ascertain a plurality of variables for calculation of a risk score associated with the perinatal care episode. The plurality of variables can include one or more of the following: a volume, a clinical severity, and a hospital service. More specifically, the variables can include or be associated with one or more of the following: a maternal age, a gestational age, a birthweight, ethnicity, a maternal risk factor, an infant risk factor, a maternal procedure, an infant procedure, a maternal lab, an infant lab, imaging, a medication, genetic testing, a diagnosis category, a CPT code, a cost of an outpatient treatment, a cost of an inpatient treatment, and a cost of an emergency room treatment. The plurality of variables can be dynamically collected and stored in one or more data sources 115.

For each of the variables, scoring module 135 determines a corresponding coefficient or weight based on the beneficiary perinatal data and optionally other information such as the historical perinatal data. As such, scoring module 135 determines a plurality of coefficients corresponding to the plurality of variables for the perinatal episode. The coefficients can be related to volume coefficients, clinical severity coefficients, hospital services coefficients, and the like. In one embodiment, the coefficients can be predetermined or preset by an operator. In another embodiment, the coefficients can be dynamically calculated using statistical algorithms, heuristic algorithms, machine-learning algorithms, and so forth.

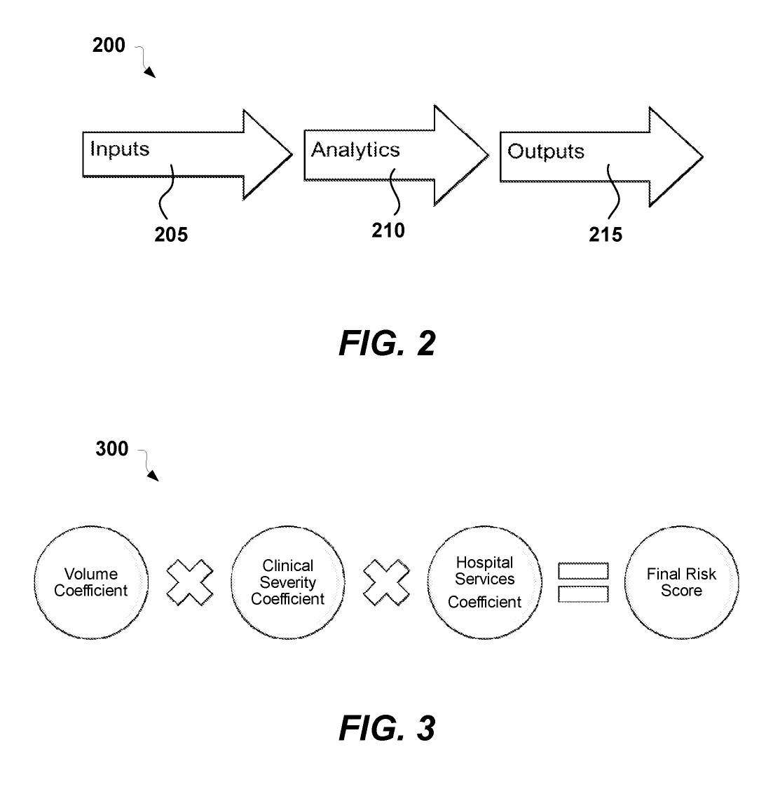

FIG. 2 represents a high-level abstract model 200 for determining the coefficients as described above. As shown in the figure, at step 205, the beneficiary perinatal data, the historical perinatal data, or related data are acquired as an input. At step 210, classification module 130 performs analytics by selectively analyzing the historical perinatal data and the beneficiary perinatal data to attribute the beneficiary to at least one perinatal care episode. At step 215, scoring module 135 ascertains the plurality of variables and determines the coefficients, which are provided as an output.

Once the coefficients are determined, scoring module 135 calculates the risk score for the beneficiary based on the coefficients. In other words, the risk score is a function of the coefficients and directly depends on the coefficients. Various methods can be employed to compute the risk score. In certain example embodiments, the risk score is calculated by multiplying two or more of the coefficients. In other embodiments, other mathematical functions can be used. FIG. 3 shows a high-level diagram 300 of calculating the risk factor according to one example embodiment. As shown in the figure, a risk factor in one given implementation is calculated by multiplying a volume coefficient, a clinical severity coefficient, and a hospital services coefficient.

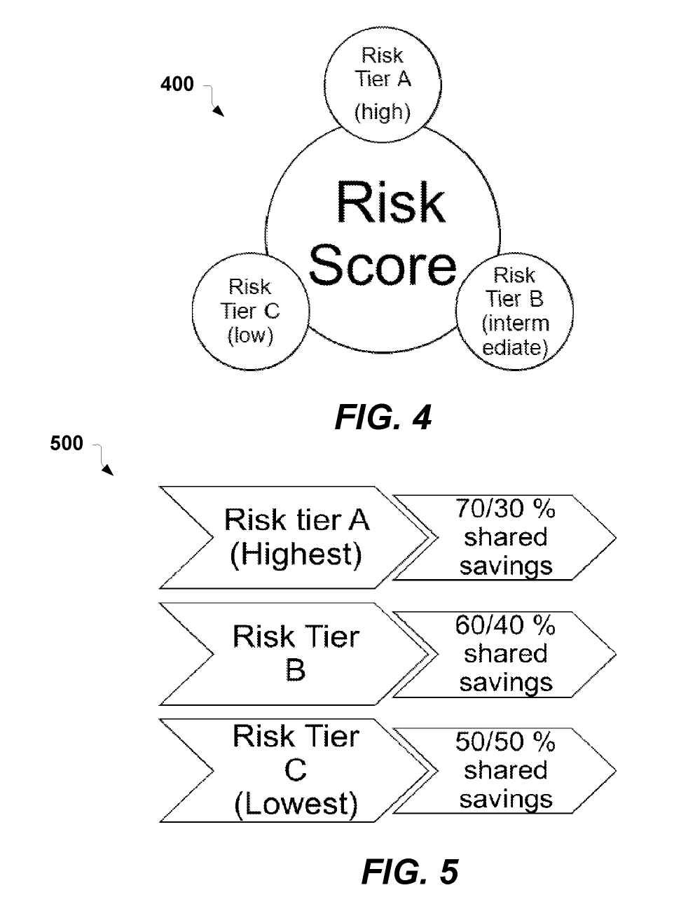

Once the risk score is calculated, scoring module 135 assigns the beneficiary a financial risk tier based on the risk score for the perinatal care episode. There can be provided two or more financial risk tiers. The financial risk tier can be selected from a plurality of risk tiers of different levels. Just one financial risk tier is assigned to the beneficiary based on the risk score. Thus, for at least one beneficiary assigned to at least one perinatal care episode, system 105 would obtain one risk score. For example, with reference to FIG. 3, when the volume coefficient is 0.45, the clinical severity coefficient is 0.35, and the hospital services coefficient is 0.75, the risk score would be 0.118. When the volume coefficient is 0.55, the clinical severity coefficient is 0.65, and the hospital services coefficient is 0.75, system 105 would provide the risk score of 0.26. When the volume coefficient is 0.85, the clinical severity coefficient is 0.75, and the hospital services coefficient is 0.95, system 105 would provide the risk score of 0.6. FIG. 4 shows a high-level representation 400 of a risk score and respective financial risk tiers A, B, and C, according to one example embodiment. FIG. 4 demonstrates that the financial risk tier (A, B, or C) can be attributed based on a particular value of calculated risk score.

Referring back to FIG. 1, system 105 further includes a payment calculation module 140, which is configured to generate cost of care data for the perinatal care episode for the beneficiary based on the financial risk tier of the beneficiary. The cost of care data can be further generated based on at least one of the following: a cost of a hospital service and a cost of a physician service. This information can be stored in one or more data sources 115. In yet additional embodiments, the cost of care data can be further based on a predetermined quality to cost ratio. Payment calculation module 140 can be further configured to estimate a payment amount for a customer based on the estimated cost of care. Here, the customer refers to at least one of the following: a health plan, a hospital system, an actuary, a provider group, or an administrator of the foregoing.

The cost of care data generated by payment calculation module 140 can be further used by payment calculation module 140 to produce a payment methodology aligned with various risk tiers. For example, clinical and financial risk tiers can be aligned and linked with payment methodology risk tiers. For these ends, for each of the final risk scores illustrated and described above, financial and payer claims data would be associated with corresponding payment methodology risk tier. FIG. 5 shows a high-level diagram 500 of three risk tiers aligned with corresponding payment methodologies according to one example embodiment.

A case scenario is provided below to explain FIG. 5. When a payer allowable charges value equals X for at least one beneficiary, a total cost of care value equals Y for that one beneficiary, and a final risk score equals Z, then the risk tier “A” (highest) would correspond to the shared savings payment methodology of 70/30% as illustrated in FIG. 5. When a payer allowable charges value equals X′ for at least one beneficiary, a total cost of care value equals Y′ for that one beneficiary, and a final risk score equals Z′, then the risk tier “B” (intermediate) would correspond to the shared savings payment methodology of 60/40% as illustrated in FIG. 5. Finally, when payer allowable charges value equals X″ for at least one beneficiary, a total cost of care value equals Y″ for that one beneficiary, and a final risk score equals Z″, then the risk tier “C” (lowest) would correspond to the shared savings payment methodology of 50/50% as illustrated in FIG. 5.

Referring again back to FIG. 1, system 105 further includes a virtual interface module 145 for providing total cost-of-care data intelligence (including reports, graphs, etc.) to system users to assist in their decision-making process. Virtual interface module 145 can include multiple screens or user interfaces that can function independently or in combination depending on user preferences. The screens and user interfaces can be presented to the system users through the client devices 110. As such, the system users can obtain the total cost-of-care data intelligence via web application, website, or web service.

In various embodiments, virtual interface module 145 is configured to receive the cost of care data, customize the cost of care data for the customer based on predetermined customer criteria to generate customized cost of care data, and generate a customized report for the customer based on the customized cost of care data. The customized report can be presented to the customer via a graphical user interface in an electronic form or as a hard copy. In addition, the customized report can include text, images, videos, animations, graphs, tables, summaries, analyses, and the like. In certain embodiments, the customized reports can also provide suggestions for improvements to a quality of care to the cost of care ratio for a value-based contract. The suggestions for improvements may be automatically generated and include information related to a lab, imaging, medication, or a procedure. In addition, virtual interface module 145 generates the customized reports such that each report can allow estimating a cost of service based on the cost of care based on a predetermined profit margin.

As mentioned above, virtual interface module 145 can provide a plurality of graphical user interfaces or screens (collectively referred to as “views”). A system user (e.g., a customer, a healthcare network administrator, or a healthcare population health administrator) can be enabled to select a desired view from the customized report. In other words, the customized report can be provided interactively, thereby enabling the system user to obtain various embodiments of the customized report in different ways. For example, the selected view can provide filtering of the customized cost of care data based on one or more of the following: a zip code, a region, a state, a specific group of beneficiaries, and at least one perinatal care episode.

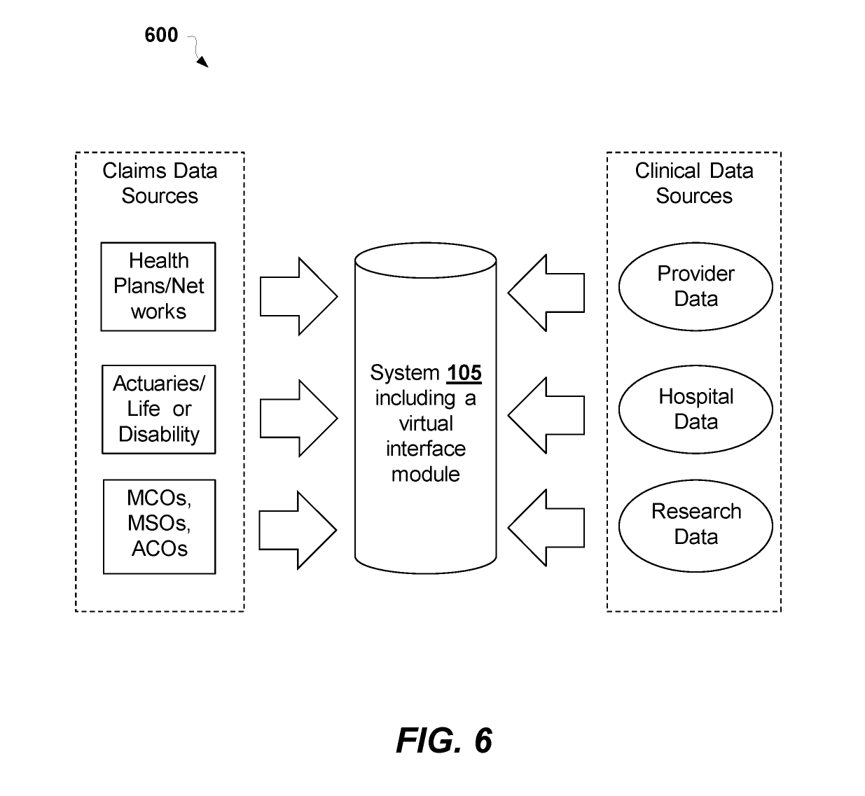

FIG. 6 represents system 105 involving data used to provide customized reports via virtual interface module 145. Virtual interface module 145 may have three or more separate interfaces to present information. They can be used independently or in combination. Each type of interface includes a unique type of metadata associated with a specifically determined data set generated via system 105 modules. In certain embodiments, there can be provided a first interface designed for a user of type A, such as a healthcare network administrator. There can be provided a second interface designed for a user of type B, such as a healthcare population health administrator. This interface can be designed to generate actionable business intelligence reports. An example customized report would encompass a perinatal period from conception to the end of first year of life. The report would also combine expanded data inputs and present information that inform the users about a comprehensive set of metrics and indicators related to cost of care reduction and improved quality of care. There can be also provided a third interface designed for a user of type C such as healthcare administrators focused on alternative payment models, value-based contracting, and utilization management. This interface may provide data intelligence reports that embody a national neonatology and perinatology virtual multi-payer value-based registry. This information would be used for decision-making or to inform development of customized alternative payment models.

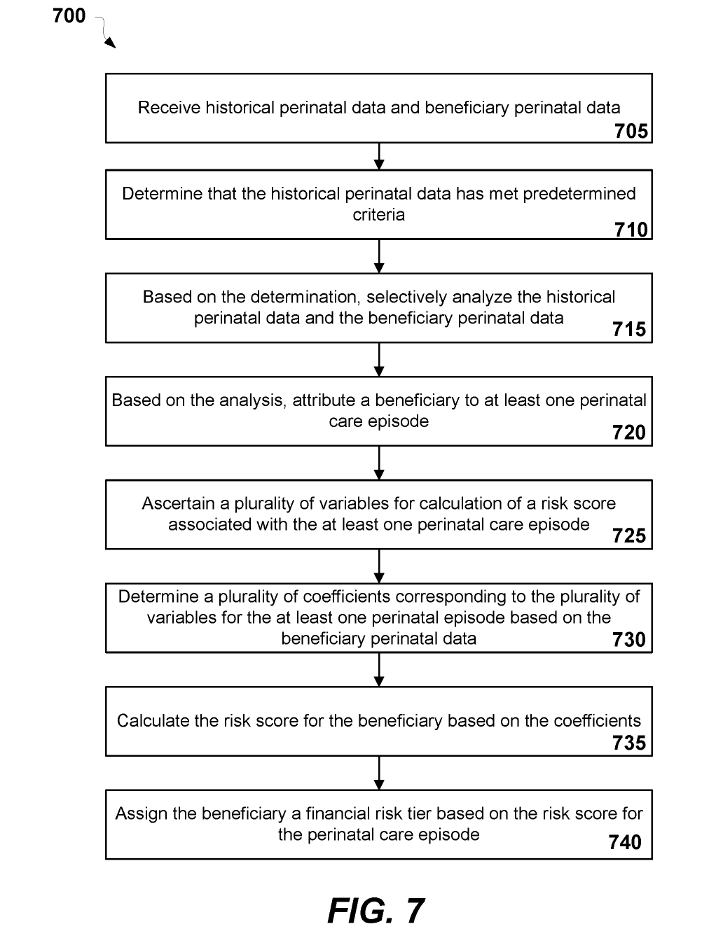

FIG. 7 is a process flow diagram showing a method 700 for estimating a cost of perinatological or neonatological care according to an example embodiment. Method 700 may be performed by processing logic that may comprise hardware (e.g., decision-making logic, dedicated logic, programmable logic, ASIC, and microcode), software (such as software run on a general-purpose computer system or a dedicated machine), or a combination of both. In one example embodiment, the processing logic refers to system 105. Below recited operations of method 700 may be implemented in an order different than described and shown in the figure. Moreover, method 700 may have additional operations not shown herein, but which can be evident for those skilled in the art from the present disclosure. Method 700 may also have fewer operations than outlined below and shown in FIG. 7.

Method 700 commences at step 705 with communication module 125 receiving historical perinatal data associated with a plurality of patients. Communication module 125 also receives beneficiary perinatal data associated with a beneficiary. The beneficiary perinatal data and historical perinatal data can be dynamically and selectively obtained from one or more data sources 115. Step 705 can be initiated automatically or upon receiving a user command to generate a customized report.

At step 710, classification module 130 determines that the historical perinatal data has met predetermined criteria, which are preset by the system user. For example, the predetermined criteria can be defined in user profiles that can be remotely modified or configured by using client devices 110. The user profiles can be stored by system 105 or data source 115.

At step 715, based on the determination, classification module 130 selectively analyzes the historical perinatal data and the beneficiary perinatal data. The analysis can include statistical analysis, comparative analysis, mapping, data correlations, modeling, validation, calibrations, and so forth. In some embodiments, machine-learning algorithms can be used.

At step 720, based on the analysis, classification module 130 attributes the beneficiary to at least one perinatal care episode. As discussed above, the perinatal care can include pre-natal care, neo-natal care, post-partum care, or pediatric care.

At step 725, scoring module 135 ascertains a plurality of variables for calculation of a risk score associated with the at least one perinatal care episode. The variables can include or be associated with one or more of the following: a maternal age, a gestational age, a birthweight, ethnicity, a maternal risk factor, an infant risk factor, a maternal procedure, an infant procedure, a maternal lab, an infant lab, imaging, a medication, genetic testing, a diagnosis category, a CPT code, a cost of an outpatient treatment, a cost of an inpatient treatment, and a cost of an emergency room treatment. The plurality of variables can be dynamically collected and stored in one or more data sources 115.

At step 730, scoring module 135 determines a plurality of coefficients corresponding to the plurality of variables for the at least one perinatal episode based on the beneficiary perinatal data. The coefficients can be related to volume coefficients, clinical severity coefficients, hospital services coefficients, and the like. The coefficients are either predetermined by the system users or operators, or dynamically calculated using statistical algorithms, heuristic algorithms, machine-learning algorithms, and so forth.

At step 735, scoring module 135 calculates the risk score for the beneficiary based on the coefficients. The risk score can be calculated as a function of the coefficients. For example, the risk score is calculated by multiplying two or more of the coefficients.

At step 740, scoring module 135 assigns the beneficiary a financial risk tier based on the risk score for the perinatal care episode. The financial risk tier can be further used to generate cost of care data for the at least one perinatal care episode for the beneficiary based on the financial risk tier of the beneficiary. As explained above, the cost of care data and financial risk tier are also used to generate customized reports for system users, which are provided via virtual interface module 145.

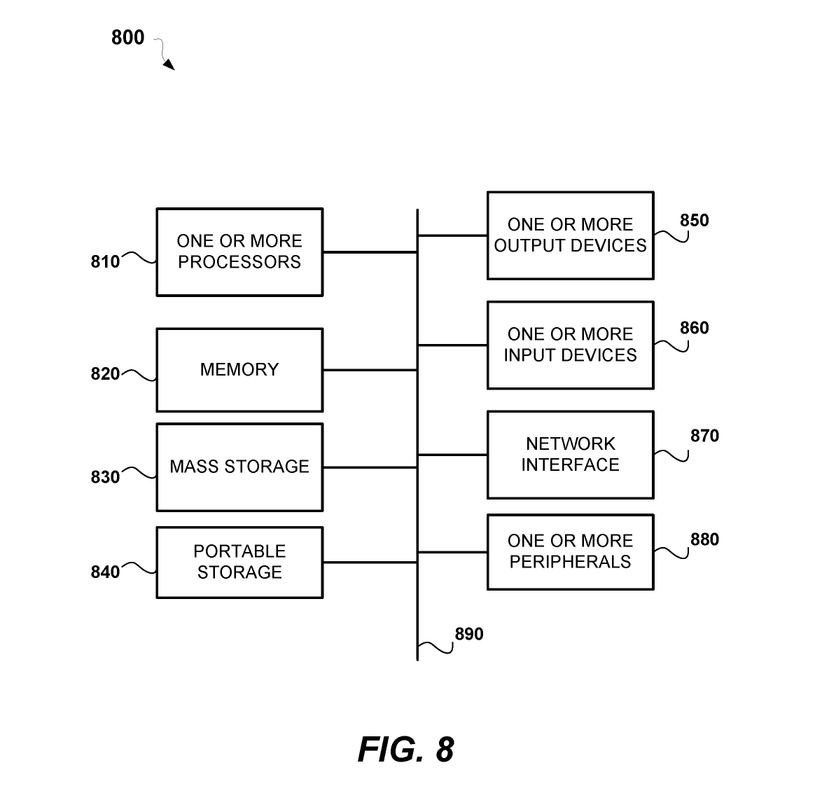

FIG. 8 is a block diagram illustrating an example computer system 800 suitable for implementing the methods described herein. In particular, computer system 800 may be an instance of system 105, client device 110, or data source 115. FIG. 8 illustrates just one example of computer system 800 and, in some embodiments, computer system 800 may have fewer elements than shown in FIG. 8 or more elements than shown in FIG. 8.

Computer system 800 includes one or more processors 810, a memory 820, one or more storage devices 830, a portable storage 840, one or more input devices 860, one or more output devices 850, network interface 870, and one or more peripherals 880. These elements can be operatively interconnected via a communication bus 890. Processors 810 are, in some examples, configured to implement functionality and/or process instructions for execution within computer system 800. For example, processors 810 may process instructions stored in memory 820 or instructions stored on storage devices 830. Such instructions may include components of an operating system or software applications.

Memory 820, according to one example, is configured to store information within computer system 800 during operation. Memory 820, in some example embodiments, may refer to a non-transitory computer-readable storage medium or a computer-readable storage device. In some examples, memory 820 is a temporary memory, meaning that a primary purpose of memory 820 may not be long-term storage. Memory 820 may also refer to a volatile memory, meaning that memory 820 does not maintain stored contents when memory 820 is not receiving power. Examples of volatile memories include RAM, dynamic random access memories (DRAM), static random access memories (SRAM), and other forms of volatile memories known in the art. In some examples, memory 820 is used to store program instructions for execution by the processors 810. Memory 820, in one example, is used by software. Generally, software refers to software applications suitable for implementing at least some operations of the methods as described herein.

Storage devices 830 can also include one or more transitory or non-transitory computer-readable storage media and/or computer-readable storage devices. In some embodiments, storage devices 830 may be configured to store greater amounts of information than memory 820. Storage devices 830 may further be configured for long-term storage of information. In some examples, the storage devices 830 include non-volatile storage elements. Examples of such non-volatile storage elements include magnetic hard discs, optical discs, solid-state discs, flash memories, forms of electrically programmable memories (EPROM) or electrically erasable and programmable memories, and other forms of non-volatile memories known in the art.

Still referencing to FIG. 8, computer system 800 may also include one or more input devices 860. Input devices 860 may be configured to receive input from a user through tactile, audio, video, or biometric channels. Examples of input devices 860 may include a keyboard, keypad, mouse, trackball, touchscreen, touchpad, microphone, one or more video cameras, image sensors, fingerprint sensors, or any other device capable of detecting an input from a user or other source and relaying the input to computer system 800 or components thereof. As such, input devices 860 can be used by users or operators of system 105 to input commands, instructions, data, settings, and the like.

Output devices 850, in some examples, may be configured to provide output to a user through visual or auditory channels. Output devices 850 may include a video graphics adapter card, a liquid crystal display (LCD) monitor, a light emitting diode (LED) monitor, an organic LED monitor, a sound card, a speaker, a lighting device, a LED, a projector, or any other device capable of generating output that may be intelligible to a user. Output devices 850 may also include a touchscreen, presence-sensitive display, or other input/output capable displays known in the art. Accordingly, output devices 850 can be used to output customized reports generated by system 105.

Computer system 800, in some example embodiments, also includes network interface 870. Network interface 870 can be utilized to communicate with external devices via one or more networks such as one or more wired, wireless, or optical networks including, for example, the Internet, intranet, local area network, wide area network, cellular phone networks (e.g. GSM communications network, packet switching communications network, circuit switching communications network), Bluetooth radio, and an IEEE 802.11-based radio frequency network, among others. Network interface 870 may be a network interface card, such as an Ethernet card, an optical transceiver, a radio frequency transceiver, or any other type of device that can send and receive information.

An operating system of computer system 800 may control one or more functionalities of computer system 800 or components thereof. For example, the operating system of computer system 800 may interact with software applications of computer system 800 and may facilitate one or more interactions between the software applications and one or more of processors 810, memory 820, storage devices 830, input devices 860, and output devices 850. The operating system of computer system 800 may interact with the software applications and components thereof. In some embodiments, the software applications may be included in the operating system of computer system 800. In these and other examples, virtual modules, firmware, or software of the software applications. In other examples, virtual modules, firmware, or software may be implemented externally to computer system 800, such as at a network location. In some such instances, computer system 800 may use network interface 870 to access and implement functionalities provided by virtual modules, firmware, or software for vehicle identification through methods commonly known as “cloud computing.”

Thus, the systems and methods for estimating costs of perinatological or neonatological care have been described. Although embodiments have been described with reference to specific example embodiments, it will be evident that various modifications and changes can be made to these example embodiments without departing from the broader spirit and scope of the present document. Accordingly, the specification and drawings are to be regarded in an illustrative rather than a restrictive sense.

What is claimed is:

1. A system for estimating a cost of perinatological or neonatological care, the system comprising:
a communication module configured to:
receive historical perinatal data associated with a plurality of patients; and
receive beneficiary perinatal data associated with a beneficiary; a classification module configured to:
determine that the historical perinatal data has met predetermined criteria;
based on the determination, selectively analyze the historical perinatal data and the beneficiary perinatal data; and
based on the analysis, attribute the beneficiary to at least one perinatal care episode; and
a scoring module configured to:
ascertain a plurality of variables for calculation of a risk score associated with the at least one perinatal care episode;
determine a plurality of coefficients corresponding to the plurality of variables for the at least one perinatal episode based on the beneficiary perinatal data;
calculate the risk score for the beneficiary based on the coefficients; and
assign the beneficiary a financial risk tier based on the risk score for the perinatal care episode.

2. The system of claim 1, wherein the determining that the historical perinatal data has met the predetermined criteria is based at least on one of the following: volume criteria, clinical hospital criteria, and clinical provider criteria.

3. The system of claim 1, wherein the plurality of coefficients includes one or more of the following: a volume, a clinical severity, and a hospital service.

4. The system of claim 1, wherein the calculating of the risk score includes multiplying two or more of the plurality of coefficients.

5. The system of claim 1, wherein the historical perinatal data and the beneficiary perinatal data are derived from at least one of the following: payer data, claims data, and clinical data.

6. They system of claim 1, further comprising a payment calculation module configured to generate cost of care data for the at least one perinatal care episode for the beneficiary based on the financial risk tier of the beneficiary.

7. The system of claim 6, wherein the payment calculation module is further configured to estimate a payment amount for a customer based on the estimated cost of care.

8. The system of claim 7, wherein the customer includes at least one of the following: a health plan, a hospital system, an actuary, and a provider group.

9. The system of claim 6, further comprising a virtual interface module configured to:
receive the cost of care data;
customize the cost of care data for the customer based on predetermined customer criteria to generate customized cost of care data; and
generate a customized report for the customer based on the customized cost of care data.

10. The system of claim 9, wherein the report provides suggestions for improvements to a quality of care to the cost of care ratio for a value-based contract.

11. The system of claim 10, wherein the suggestions for improvements include information related to one or more of the following: a lab, imaging, a medication, and a procedure.

12. The system of claim 9, wherein the report allows estimating a cost of service based on the cost of care based on a predetermined profit margin.

13. The system of claim 9, wherein a user is to select a desired view from the customized report.

14. The system of claim 13, wherein the selected view is to filter the customized cost of care data based on one or more of the following: a zip code, a region, a state, a specific group of beneficiaries, and the at least one perinatal care episode.

15. The system of claim 13, wherein the user is one of the following: a healthcare network administrator and a healthcare population health administrator.

16. The system of claim 6, wherein the generating of the cost of care data is further based on at least one of the following: a cost of a hospital service and a cost of a physician service.

17. The system of claim 6, wherein the generating of the cost of care data is further based on a predetermined quality to cost ratio.

18. The system of claim 1, wherein the financial risk tier is selected from a plurality of risk tiers of different levels.

19. The system of claim 1, wherein the communication module is agnostic to a data source associated with the historical perinatal data and the beneficiary perinatal data when formatted according to an industry accepted format.

20. The system of claim 1, wherein the perinatal care data include one or more of the following: a pre-natal care, a neo-natal care, a post-partum care, and a pediatric care.

21. The system of claim 1, wherein the plurality of variables includes one or more of the following: a maternal age, a gestational age, a birthweight, ethnicity, a maternal risk factor, an infant risk factor, a maternal procedure, an infant procedure, a maternal lab, an infant lab, imaging, a medication, genetic testing, a diagnosis category, a Current Procedural Terminology (CPT) code, a cost of an outpatient treatment, a cost of an inpatient treatment, and a cost of an emergency room treatment.

22. A method for estimating a cost of perinatological or neonatological care, the method comprising:
receiving, by a communication module, historical perinatal data associated with a plurality of patients;
receiving, by the communication module, beneficiary perinatal data associated with a beneficiary;
determining, by a classification module, that the historical perinatal data has met predetermined criteria;
based on the determination, selectively analyzing, by the classification module, the historical perinatal data and the beneficiary perinatal data;
based on the analysis, attributing, by the classification module, the beneficiary to at least one perinatal care episode;
ascertaining, by a scoring module, a plurality of variables for calculation of a risk score associated with the at least one perinatal care episode;
determining, by the scoring module, a plurality of coefficients corresponding to the plurality of variables for the at least one perinatal episode based on the beneficiary perinatal data;
calculating, by the scoring module, the risk score for the beneficiary based on the coefficients; and
assigning, by the scoring module, the beneficiary a financial risk tier based on the risk score for the perinatal care episode.

23. A system for estimating a cost of perinatological or neonatological care, the system comprising:
a communication module configured to:
receive historical perinatal data associated with a plurality of patients; and
receive beneficiary perinatal data associated with a beneficiary;
a classification module configured to:
determine that the historical perinatal data has met predetermined criteria;
based on the determination, selectively analyze the historical perinatal data and the beneficiary perinatal data; and
based on the analysis, attribute the beneficiary to at least one perinatal care episode; and
a scoring module configured to:
ascertain a plurality of variables for calculation of a risk score associated with the at least one perinatal care episode;
determine a plurality of coefficients corresponding to the plurality of variables for the at least one perinatal episode based on the beneficiary perinatal data;
calculate the risk score for the beneficiary based on the coefficients;
assign the beneficiary a financial risk tier based on the risk score for the perinatal care episode;
a payment calculation module configured to generate cost of care data for the at least one perinatal care episode for the beneficiary based on the financial risk tier of the beneficiary; and
a virtual interface module configured to:
receive the cost of care data;
customize the cost of care data for the customer based on predetermined customer criteria to generate customized cost of care data; and
generate a customized report for the customer based on the customized cost of care data.
